(19) United States
(12) Patent Application Publication
(10) Pub. No.: US 2017/0258325 A1
(43) Pub. Date: Sep. 14, 2017

(54) PORTABLE HEAD-MOUNTED OCULAR-VESTIBULAR TESTING SYSTEM

(71) Applicant: Carrick Technologies, LLC, Cape Canaveral, FL (US)

(72) Inventors: Frederick Robert Carrick, Cape Canaveral, FL (US); Rosemary Frances Kitchiner, Milton Keynes (GB); Paul James Toland, Milton Keynes (GB)

(73) Assignee: Carrick Technologies, LLC, Cape Canaveral, FL (US)

(21) Appl. No.: 15/343,411

(22) Filed: Nov. 4, 2016

Related U.S. Application Data
(60) Provisional application No. 62/307,756, filed on Mar. 14, 2016.

(51) Int. Cl. A61B 3/113; A61B 5/00; A61B 3/00; A61B 3/14

(52) U.S. Cl. CPC A61B 3/113; A61B 3/145; A61B 5/4863; A61B 5/4023; A61B 3/0025; A61B 3/0041

(57) ABSTRACT

A portable, miniaturized, lightweight ocular-vestibular testing unit with sensors in a head-mounted unit and processor and battery units either incorporated into the head-mount or on other parts of the body, such as a waist belt and connecting to one or more displays via wireless local network. A digital camera for each eye and multiple sensors are configured for ocular and vestibular system testing in a wide variety of positions and locations of a user. The unit records data from the digital cameras and sensors locally to the unit. Recorded data may be viewed via a wireless local network data connection on external displays.

### CROSS REFERENCE TO RELATED APPLICATIONS

The present application claims priority to U.S. Provisional Patent Application No. 62/307,756 entitled Portable Head-Mounted Ocular-Vestibular Testing System, which was filed on Mar. 14, 2016, the contents of which is incorporated by reference herein in it's entirety.

### TECHNICAL FIELD

The present disclosure relates generally to the use of sensors in the field of neurological testing. More particularly, the present application relates to the use of a physically distributed system of miniaturized sensors coupled with information and image processing systems to achieve neurological testing.

### BACKGROUND

The study of eye movements provides useful data to investigate the working of the brain; both normal function and pathologies. Over the past 60 years, eye movements have been analyzed to provide insights into disorders ranging from muscular dystrophy to autism.

Originally, data on eye movements was mapped using coils placed on the eye surface. Major advances in electronics have led to the use of video recording and analysis, which can produce equally accurate and more visually appealing and recordable results.

In the field of functional neurology, eye movements are now routinely used to detect and diagnose brain and vestibular abnormalities and to provide measurement of the results of therapies. For example, distinct audible tones played to the patient have been shown to affect the ocular and vestibular systems. Further, the position of the head in 3 dimensions has been shown to affect the ocular and vestibular systems as well.

Electronystagmography (“ENG”) is a diagnostic test that records involuntary movements of the eye caused by a condition known as nystagmus. Nystagmus is a condition of involuntary or voluntary (rarely) eye movement usually manifesting itself in infancy that can result in limited or impaired vision. Nystagmus is commonly known as “dancing eyes.”

The vestibular system is the sensory system that most largely contributes to a person's sense of balance and spatial orientation for the purpose of coordinating movement with balance. ENG has been widely used to diagnose the cause of vertigo, dizziness or balance dysfunction by testing the vestibular system.

The ENG test is performed by attaching electrodes around the patient's nose and measuring the movements of the eye in relation to the ground electrode. The vestibular system monitors the position and movements of the head to stabilize retinal images. That information is integrated with the visual system and spinal afferents in the brain stem to produce the vestibule-ocular reflex (“VOR”).

ENG provides an objective assessment of the oculomotor and vestibular systems. Infrared (“IR”) video systems provide a newer standard for recording eye movement in the ENG context by allowing for a more detailed observation and analysis of these eye movements. That system is known as video nystagmography (“VNG”) or more recently as video oculography (“VOG”).

A similar test is performed for testing vertigo by using the caloric reflex test, which can be induced by air or water of specific temperatures, typically ±7 degrees Celsius from body temperature, irrigated into the external auditory canal.

The standard ENG test battery consists of three parts: (1) oculomotor evaluation; (2) positioning and positional testing; and (3) caloric stimulation of the vestibular system. The comparison of results obtained from various subtests of ENG assists in determining whether a disorder is central or peripheral. In peripheral vestibular disorders, the side of lesion can be inferred from the results of caloric stimulation and, to some degree, from positional findings.

VOG testing provides the newest standard protocol many healthcare providers are currently implementing for a variety of tests including, but not limited to, inner ear functions. Contrasted with ENG, VOG measures the movements of the eyes directly through infrared cameras, instead of measuring the mastoid muscles around the eyes with electrodes like the previous ENG versions. Accordingly, VOG testing is more accurate, more consistent, and more comfortable for the patient. By having the patient more comfortable and relaxed, the practitioner can more easily attain consistent and accurate test results.

VOG testing can be used to determine if an inner-ear disease could be the cause of dizziness symptoms or balance issues. One of the most salient advantages of VOG testing is that it allows the practitioner to distinguish between a unilateral (one ear) and bilateral (both ears) vestibular loss. VOG comprises a series of tests designed to document a person's ability to follow visual objects with his or her eyes and how well the eyes respond to information from the vestibular system.

The VOG test is also capable of addressing and measuring: (1) the functionality of each ear; and (2) if a vestibular deficit may be the cause of the symptoms the patient presents. In order to follow the eyes' movement, the practitioner encases the patient's eyes in infrared goggles. That allows the movement of the patient's eyes to be recorded during VOG testing.

ENG or VOG can be used to record nystagmus during oculomotor tests such as saccades, pursuit and gaze testing, optokinetics and also calorics (dithermal or monothermal). Abnormal oculomotor test results may indicate either systemic or central pathology as opposed to peripheral (vestibular) pathology. Optokinetics generally are used as a cross-check on abnormal responses to oculomotor tests. Both of these tests use a “light bar” involving a moving light (usually red) which the patient will track with the eyes.

The caloric irrigation is the only vestibular test that allows testing of the vestibular organs individually. However, the caloric irrigation vestibular test only tests one of the three semicircular canals: the horizontal canal.

While VOG is the most widely used clinical laboratory test to assess vestibular function, normal VOG test results do not necessarily mean that a patient has typical vestibular function. VOG abnormalities can be useful in the diagnosis and localisation of the site of lesion; however, many abnormalities are non-localizing; therefore, the clinical history and otologic examination of the patient are vital in formulating a diagnosis and treatment plan for a patient presenting with dizziness or vertigo.

Numerous apparatus and methods have previously been designed for testing and assessment of vestibular and oculomotor reflexes and function. The previously known systems generally include non-portable equipment that is cumbersome for clinicians and patients top use. For example, U.S. Patent Application 2010/0036289 by White, et al., discloses a rotary disk apparatus capable of inducing vestibule-ocular reflex eye movement by securely rotating a patient. The rotary disk apparatus includes bulky headgear attached to video recording equipment, a rotating platform, and cabled data gathering and transmitting means. In order to use the rotary disk apparatus, a patient sits or lays down on the platform, which is then rotated for a short period of time to induce nystagmus. This requires specific and restricting: (1) patient positioning and securing; (2) rotational platform speeds and duration; (3) rotational power generating means; and (4) bulky camera equipment and recording equipment strapped to the user's head, shoulders and torso.

U.S. Pat. No. 8,529,463 to Della Santina et al. illustrates another apparatus and method for testing vestibular and oculomotor function. The apparatus includes (1) a curved track supported by a plurality of bearings; (2) an engine configured to selectively displace the track; (3) a head coupling component coupled to the track; and (4) a sensor capable of measuring and reporting motion of the engine, the track and/or the user's head. The head coupling component is configured to convey a movement generated by the engine to a subject's head in one or more axes. To do so, the head coupling component rotates the user's head about an axis outside the device and through the user's cranio-cervical junction region. The system is not portable, it is cumbersome and requires elaborate equipment and set up steps.

### SUMMARY

Objects, advantages and novel features, and further scope of applicability of the present invention will be set forth in part in the detailed description to follow, taken in conjunction with the accompanying drawings. The objects, advantages and novel features, and further scope of applicability of the present invention will become apparent to those skilled in the art upon examination of the following, or may be learned by practice of the invention. The objects and advantages of the invention may be realized and attained by means of the instrumentalities and combinations particularly pointed out in the appended claims.

Aspects of the present disclosure include a distributed physical system of device components with a miniaturized, portable, device comprising head-mounted miniaturized sensors coupled with information and image processing systems that monitors eye movements, movements of the head in three dimensions and audible tones to perform non-invasive testing of neurological functions and deficits.

The disclosed ocular-vestibular testing system provides recording of selected inputs (eye position, head tilt in 3 dimensions), together with selected automated stimulus (stationary dots, moving patterns) to elicit specific responses. Those responses then indicate the operation of the ocular and vestibular systems and the neurological pathways that drive those systems. In addition, the clinician may use other, external stimuli (moving finger following etc.) to elect specific responses.

The system described in the present application provides an ocular-vestibular testing apparatus and method that is: (1) portable; (2) completely self-contained, requiring no cables to external equipment; and therefore (3) useful in a variety of situations and modalities. The system is divided into three major components:
a. sensors (eye and position) which would always be mounted on a user's head
b. processing and storage components which can either be co-mounted with the sensors on the user's head, or mounted elsewhere on the patient body, such as on a waist belt
c. battery power component which can either be co-mounted with the sensors on the user's head, or alternatively mounted elsewhere on the patient body such as on a waist belt, either with the processing component or separately.

The system described in the present application also provides an adjustable head-mount base, which allows use on a variety of head shapes and sizes, suitable for use by adults and children alike. In an alternative embodiment, the system can be used by mechanically connecting it to a table or frame mounted head support. In a further embodiment, the head mounted unit contains only the cameras and sensors (and hence is smaller than the composite unit) and is connected by a cable to a unit mounted elsewhere on the body, such as a waist belt, containing the processor units and storage, thus preserving the portability of the system.

The present system is powered by a rechargeable 5V USB power pack. Accordingly, the disclosed apparatus and method does not require any external power cabling or any other mechanical power transmission means during normal operation. The location of the USB power pack for a complete head-mounted system is also advantageous for it is located at the rear of the head-mount base, thus counter-balancing the camera and processing units located at the front of the system. That counterbalancing results in a less cumbersome and more comfortable set up for the user than previously known systems. Again, in an alternative embodiment, the power pack would be mounted elsewhere on the body such as waist-mounted.

The disclosed apparatus includes at least two IR digital cameras (one for each eye) that record the position of the user's eyes in real time. That allows the production of real-time video recording together with analysis of the position of the pupil of each eye using digital analysis. Multiple IR light emitting diodes (“LED”) are provided within the eye enclosures for camera illumination, which enhances the detection of the eye pupil.

The disclosed apparatus also includes adjustable holders positioned over each of the user's eyes. In an illustrative embodiment, the holders comprise opaque caps fitted to enable testing of the eye position in complete darkness. An alternative embodiment includes a colored cap that can be fitted to test eye responses under different lighting conditions. The disclosed apparatus may be configured in such a way that each eye enclosure may have different caps fitted at any given time.

The disclosed apparatus also provides a six degree of freedom (6DOF) reading, with the sensors being positioned on each side of the head to provide differential readings. That configuration allows the present system to record the position of the head in the X, Y & Z axes, using dual inertial gyroscopes or accelerometers.

The disclosed system also includes a real-time clock that synchronizes the data recording for all inputs. That way the viewing of an automated stimulus can be shown against the resulting eye or head positions.

### BRIEF DESCRIPTION OF THE DRAWINGS

The accompanying drawings, which are incorporated into and form a part of the specification, illustrate an embodiment of the present invention and, together with the description, serve to explain the principles of the invention. The drawings are only for the purpose of illustrating various embodiments of the invention and are not to be construed as limiting the invention.

FIG. 1 is a frontal view of the disclosed ocular-vestibular testing apparatus fitted on a user's head according to aspects of the present disclosure.

FIG. 2 is a right-side view of the disclosed ocular-vestibular testing apparatus fitted on a user's head according to an aspect of the present disclosure.

FIG. 3 is a right-side view of an alternative embodiment of the disclosed ocular-vestibular testing apparatus fitted on a user's head.

FIG. 4 is a schematic illustration of an embodiment of the disclosed ocular-vestibular testing apparatus in wireless communication with wireless display devices via a wireless network according to aspects of the present disclosure.

FIG. 5 is a schematic block diagram illustration of a processing unit configured for controlling an ocular-vestibular testing apparatus according to aspects of the present disclosure.

FIG. 6 is an illustration of a split embodiment configured with sensors on a head mounted unit and processing unit and battery mounted remotely on a waist belt.

### DETAILED DESCRIPTION

Aspects of the present disclosure include a portable miniaturized, head-mounted, lightweight ocular-vestibular testing assembly that provides recording of selected inputs, the selected inputs comprising eye position and head tilt in 3 dimensions, together with selected automated stimulus. The automated stimulus may include stationary dots and/or moving patterns, to elicit specific responses that indicate the operation of the ocular and vestibular systems and the neurological pathways that drive those systems. The visual stimulus is configurable by the clinician.

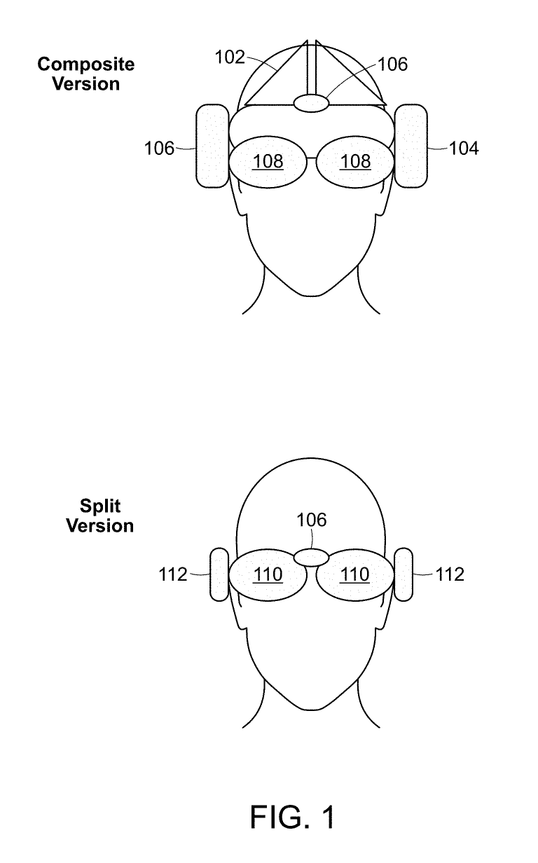

Referring to FIG. 1, the assembly includes an adjustable head-mount base 102 designed to fit securely to a user's head. The diagram shows two alternative configurations; the top (composite version) being with all components being headed mounted; the lower (split version) showing a base just containing the sensor components, with the processing and battery components being housed in a remote waist belt (not shown). The head mount base 102 may include adjusting members so it can be fitted to different head sizes and shapes. The base 102 includes a front portion, two side portions and a back portion, in which the orientation of the front portion of the base coincides with an orientation of the user's face.

The composite version assembly also includes a processing unit 104 coupled to the base unit 102. In an illustrative embodiment, the processing unit includes a left and right eye digital infrared (IR) camera located on the front portion of the base 102. Each camera may include associated controller boards. The processing unit 104 may be connected to the base unit by a flexible coupling member 106. The split version assembly includes a simpler head mount 110, resembling a large pair of glasses, with a sensor unit 112 which connects to a remote processing and battery assembly (not shown).

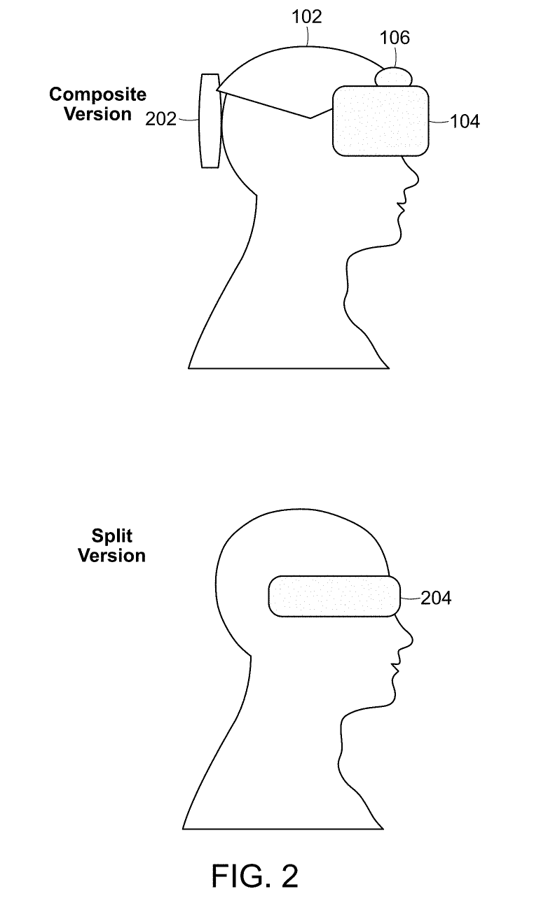

Referring to FIG. 2, again showing two configurations: composite and split versions. In the composite version, a power supply unit 202 is connected to the back portion of the base. In a particular implementation, the power supply unit 202 may be a 5-volt universal serial bus rechargeable battery pack, for example. In the illustrative embodiment, the power supply unit 202 counter-balances the camera and processing units 104 located at the side portions of the system. The power supply unit 202 is electrically coupled to and provides power to the controller boards in the processing unit 104. In the split version, the simpler head mount 204 only contains the sensors, with the processing unit and battery mounted remotely on a waist belt (not shown). In the illustrative embodiment, at least two infrared digital cameras are located each over one of the user's eyes. The cameras record the position of the user's eyes in real time. This allows the production of real-time video recording together with digital analysis of the position of the pupil of each eye.

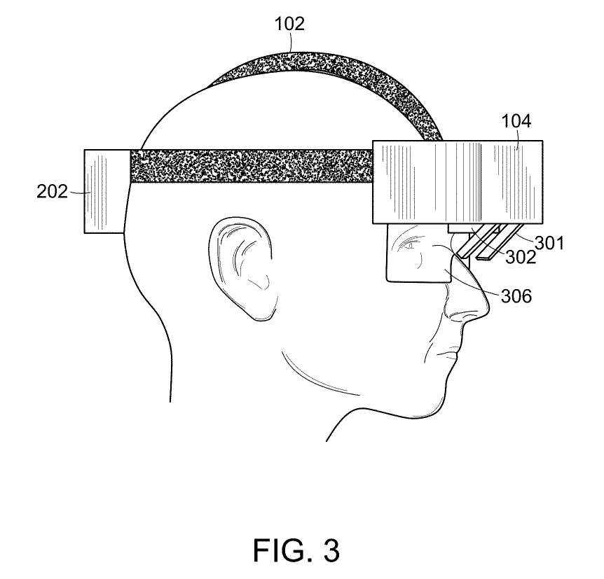

Referring to FIG. 3, in an illustrative embodiment the digital cameras 302 are positioned on the processing unit 104 to view the area including the user's eyes via infrared reflective mirrors without obstructing the field of view of the user's eyes. Eye shields 306 extending from the processing unit 104 may be provided for positioning and support of the processing unit 104.

According to another aspect of the present disclosure, the apparatus also includes at least two eye enclosures 108 (FIG. 1). Each of the eye enclosures 108 include multiple IR light emitting diodes, which provide camera illumination.

According to another aspect of the present disclosure, adjustable holders are positioned over each of the user's eyes. The adjustable holders include interchangeable caps. The interchangeable caps may include opaque caps, which enable testing of the user's eye position in complete darkness, and tinted caps, which enable testing of the user's eye position under conditions of a selected frequency of visible light, for example.

A real-time clock is configured to synchronize the data recording for the various inputs. This enables a view of an automated stimulus to be displayed in conjunction with a display of the resulting eye or head positions.

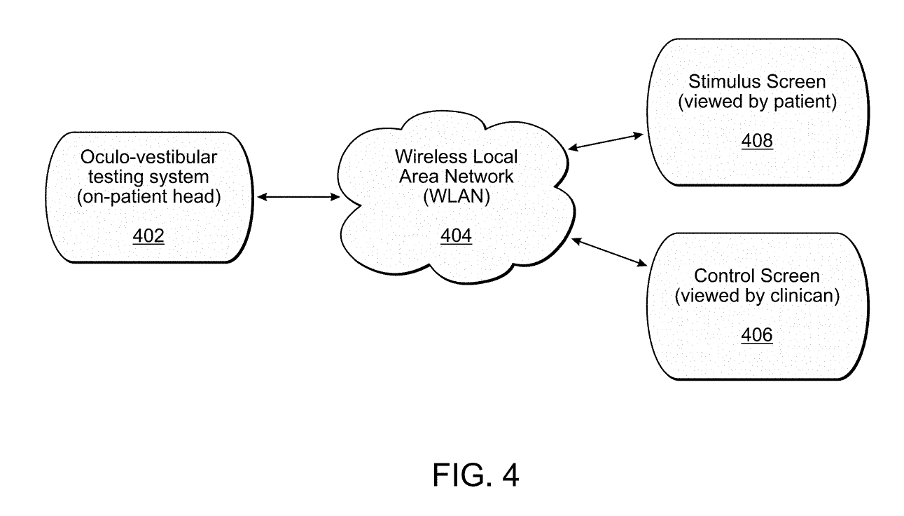

In an illustrative embodiment, the apparatus also includes a wireless local area network data connection using the IEEE 802.11 standard for communicating with a wireless display device. Referring to FIG. 4, in an illustrative embodiment two types of displays may be coupled to the disclosed ocular-vestibular testing system 402 via a wireless local area network 404. A control display 406 may be coupled in wireless communication with the ocular-vestibular testing system 402 via the wireless local area network 404 and used by a clinician to initiate start of stimulus actions and the viewing of resultant video and charts, for example. A stimulus display 408, may also be coupled in wireless communication with the ocular-vestibular testing system 402 via the wireless local area network 404 to display selected stimuli for viewing viewed by the user.

According to aspects of the present disclosure, the control display 406 and the stimulus display 408 each include a web browser supporting the HTTP standard. This avoids the need to for downloading additional software to the control display 406 and stimulus display 408. The control displays 406 and stimulus display 408 can each include a computer running Windows, Apple or Linux operating systems, a smart phone, a tablet computer, or a smart TV display, for example.

In addition to or alternative to the stimulus display 408, the system may include a set of three laser dot projectors, which can show bright dots against a suitable background in front of the patient. Stimulus actions include a variety of stimulus types, such as stationary and moving dots, stationary and moving patterns. Stimulus actions can be pre-programmed sequences or random sequences, both of which may be recorded during a testing session.

Resulting eye and head positions, together with the stimuli that evoked them, may be charted in a variety of formats and displayed in real-time on the control display. Previously recorded data may also be displayed in real-time, slow-speed or accelerated-speed to aid the clinician in diagnosis.

The gathered and recorded data is downloadable to an external device for long-term storage or further analysis. Data may be downloaded in a variety of standard formats, such as CSV, XLS, XML. The disclosed system may also include a security module to ensure that all data may be protected from un-approved access, including access via the WLAN.

In an illustrative embodiment, the apparatus includes three laser diodes configured to project bright dots on a vertical surface in front of the user. The laser diodes may be controlled from the processing unit to show 0, 1, 2 or 3 dots at a time as a stimulus, for example.

Embodiments of the disclosed ocular-vestibular testing assembly may also include a stereo audio output to receive headphones for audible stimulus in the form of tones to be listened to by the system's user during testing.

In an illustrative embodiment, the ocular-vestibular testing assembly may also include an enclosure for each eye. The enclosures are configured to provide a light seal against the user's face. The enclosures may also position the digital cameras to view the area including the user's eyes via infrared reflective mirrors without obstructing the field of view of the user's eyes.

The disclosed ocular-vestibular testing assembly of may also include multiple opaque holders to attach the closed caps so that they occlude external light, or colored lenses for testing sensitivity to different frequencies of visible light. Each eye enclosure is capable of holding a different cap or lens compared to the opposite eye enclosure.

According to aspects of the present disclosure, the disclosed ocular-vestibular testing assembly may also include inertial gyroscopic and accelerometer sensors connected to the processing controller boards and providing data showing the X, Y and Z axis positions, with a 6 DOF (degree of freedom) sensor on each side of the processing unit to provide differential readings.

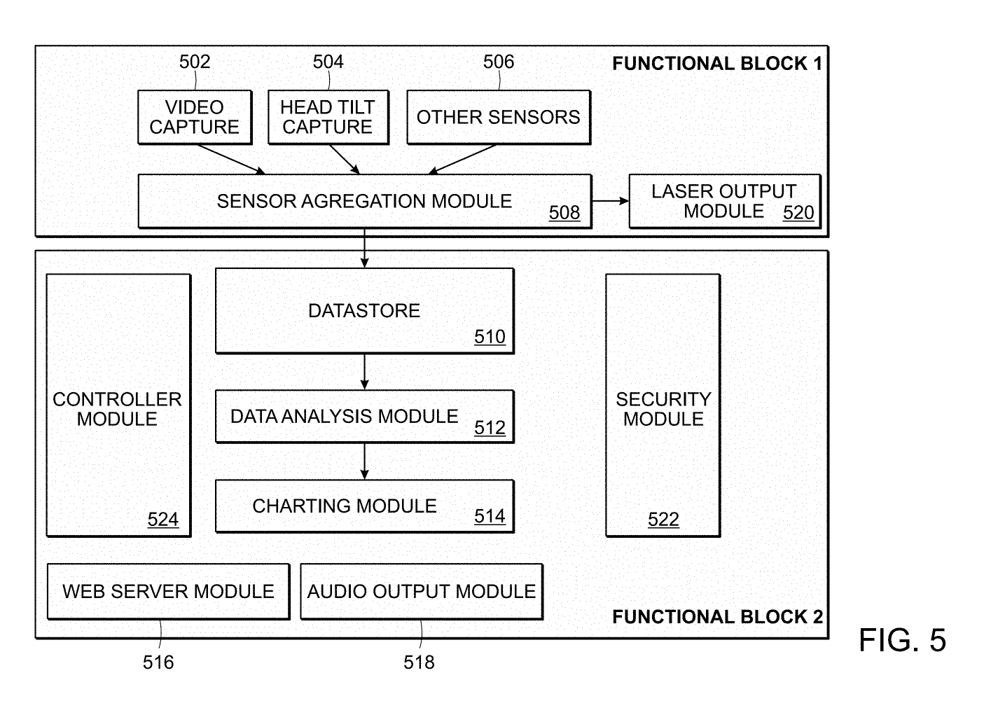

FIG. 5 is a schematic block diagram illustration of a processing unit configured for controlling an ocular-vestibular testing apparatus according to aspects of the present disclosure. The block diagram is divided into two major sections: Functional Block 1 containing the sensor components; and Functional Block 2 containing the processing and storage components. This division supports the alternative configuration, with either all components being head-mounted, or the sensors being head-mounted and other components being mounted remotely on a waist belt. The processing unit 500 may include a video capture module 502, a head tilt capture module 504 and other sensors 506 in communication with a sensor aggregation module 508. The sensor aggregation module 508 aggregates input from the video capture module 502, a head tilt capture module 504 and other sensors 506 for storage in a data storage module 510. A data analysis module 512 analyses data in the data storage module 510 and co-operatively with a charting module 514 generates output for displaying to user, displaying to an operator and/or for controlling the ocular-vestibular testing apparatus. A web server module 516, may be configured as a web server for communicating wirelessly with external devices such as displays 406, 408, for example. An audio output module 518 may be configured for generating audio stimulus for output to user, via an audio port, for example. A laser output module 520 may be configured for generating visual stimulus for presentation to a user. A security module 522 may be configured to ensure that user data is be protected from un-approved access, including access via the WLAN, for example. A controller module 524 may be configured for controlling the ocular-vestibular testing apparatus to initiate and terminate testing, select visual and/or audible stimulus to be presented to a user, and control recording of testing video data, for example.

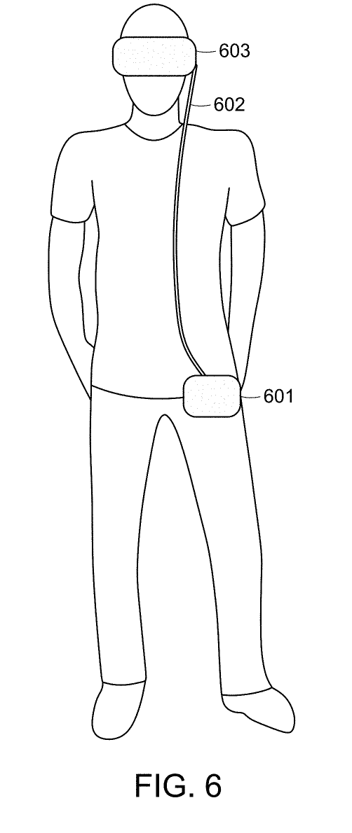

FIG. 6 is an illustration of a configuration with the processing and battery components 601 mounted remotely on a waist belt and connected via a cable 602 to a simplified head-mount sensor component 603.

What is claimed is:

1. A portable ocular-vestibular testing apparatus, comprising:
a wearable mounting structure configured for fitting onto a user's head;
a first [IR] camera directed toward a first eye of the user;
a second [IR] camera directed toward a second eye of the user;
a motion sensor configured for tracking movement of the user's head;
a processor module attached to the wearable mounting structure
a battery unit attached to the wearable mounting structure and coupled to the processor module, the battery unit configured for counter-balancing the processor module
wherein the processor module comprises:
camera controller circuitry coupled to the first camera and to the second camera, and
processing circuitry coupled to the first camera, the second camera, and the motion sensor, the processing circuitry including memory for storing image data received from the first camera and from the second camera and for storing motion signals received from the motion sensor.

2. The apparatus of claim 1, wherein the processing circuitry includes clock circuitry for time stamping the image data and the motion signals.

3. The apparatus of claim 1, wherein the first camera and second camera are mounted to the processor module.

4. The apparatus of claim 1, wherein the motion sensor is mounted to the processor module.

5. The apparatus of claim 1, wherein the processing circuitry is configured to detect the user's pupil positions in real time based on the image data.

6. The apparatus of claim 5, wherein the processing circuitry is configured to generate a video rendering of the user's eye movements based on the image data.

7. The apparatus of claim 6, wherein the processing circuitry is configured to record the video rendering.

8. The apparatus of claim 6, wherein the processing circuitry is configured to record eye positions of the user and simultaneous head tilt of the user in 3 dimensions based on the image data and the motion signals.

9. The apparatus of claim 6, wherein the processing circuitry is configured to include an indication of the user's pupil positions in the video rendering of the user's eye movements.

10. The apparatus of claim 5, wherein the processor module comprises a stimulus projecting device configured to present a predetermined visual stimulus to the user.

11. The apparatus of claim 10 wherein the stimulus projecting device includes laser diodes directed to a vertical surface in front of the user, wherein the processing circuitry is configured to control the laser diodes.

12. The apparatus of claim 10, wherein the processing module includes an audio output port and wherein the processing circuitry is configured to communicate an audible stimulus signal to the audio output port, wherein the audible stimulus is synchronized with the predetermined visual stimulus.

13. The apparatus of claim 10, wherein the predetermined visual stimulus is predetermined to elicit specific responses that indicate operation of the user's ocular and vestibular systems and neurological pathways.

14. The apparatus of claim 13 wherein the predetermined visual stimulus comprises a plurality of stationary dots and moving patterns.

15. The apparatus of claim 10, wherein the processing circuitry comprises wireless transceiver circuitry configured for wireless communication of the video rendering to a separate external display device.

16. The apparatus of claim 15, wherein the wireless transceiver circuitry is configured for receiving control signals from the separate external display device.

17. The apparatus of claim 16, wherein the control signals include a first signal configured to cause the processing circuitry to start recording of the video rendering and a second signal configured to cause the processing circuitry to stop recording of the video rendering.

18. The apparatus of claim 17, wherein the control signals include a third signal configured to cause the processing circuitry to start presenting the video stimulus and a fourth signal configured to cause the processing circuitry to stop presenting the video stimulus.

19. The apparatus of claim 17, wherein the separate external display device is in the group consisting of a mobile computing device, smart phone, personal computer and tablet computer.

20. The apparatus of claim 5, wherein the processing circuitry comprises wireless transceiver circuitry configured for wireless communication of a predetermined visual stimulus image to a separate external display device, wherein the predetermined visual stimulus image is predetermined to elicit specific responses that indicate operation of the user's ocular and vestibular systems and neurological pathways.

21. The apparatus of claim 20 wherein the wireless transceiver circuitry comprises a wireless data module configured to communicate in compliance with the IEEE 802.11 standard

22. The apparatus of claim 1, wherein the cameras are IR cameras.

23. The apparatus of claim 22, further comprising an eye enclosure covering each eye of the user, the eye enclosure comprising an IR light source configured for illuminating a viewing area of a corresponding one of the IR cameras.

24. The apparatus of claim 23 further comprising a second eye enclosure covering each eye of a user, the second eye enclosure providing a light seal against the user's face and positioning the digital cameras to view the user's eyes via infrared reflective mirrors without obstructing the field of view of the user's eyes.

25. The apparatus of claim 23, further comprising an adjustable holder positioned over each eye of the user, the adjustable holder including an interchangeable cap configured for covering a corresponding one of the user's eyes.

26. The apparatus of claim 25 wherein the interchangeable cap is opaque for testing the user's eye position under conditions of darkness.

27. The apparatus of claim 25, wherein the interchangeable cap comprises a colored lens for testing the user's eye position under exposure to a selected frequency of visible light.

28. The apparatus of claim 1 wherein the motion sensor comprises inertial gyroscopic and accelerometer sensors generating the motion signals, the motion signals including X, Y and Z axis positions.

29. The apparatus of claim 28, wherein the motion sensor comprises a six degree of freedom sensor on each side of the processing unit to provide differential readings.

30. A portable ocular-vestibular testing apparatus, comprising:
a sensor module configured for fitting onto a user's head;
a first [IR] camera directed toward a first eye of the user;
a second [IR] camera directed toward a second eye of the user;
a motion sensor configured for tracking movement of the user's head;
a processor module configured for mounting to a first wearable article configured for wearing on a user's body;
a battery unit configured for mounting to a second wearable article configured for wearing on a user's body, the battery unit coupled to the processor module;
wherein the processor module comprises:
camera controller circuitry coupled to the first camera and to the second camera, and
processing circuitry coupled to the first camera, the second camera, and the motion sensor, the processing circuitry including memory for storing image data received from the first camera and from the second camera and for storing motion signals received from the motion sensor.
